(19) United States
(12) Patent Application Publication
(10) Pub. No.: US 2014/0296155 A1
(43) Pub. Date: Oct. 2, 2014

(54) ANTI-STRETCH MARK SKIN CREAM AND METHOD FOR MAKING AND USING THEREOF

(71) Applicants: Amy NEWBURGER, (US); Konstantinos M. LAHANAS, (US)

(72) Inventors: Amy Newburger, Greenwich, CT (US); Konstantinos M. Lahanas, Paramus, NJ (US)

(21) Appl. No.: 14/346,542

(22) PCT Filed: Sep. 24, 2012
(86) PCT No.: PCT/US2012/056951

§ 371 Date: Mar. 21, 2014

Related U.S. Application Data
(60) Provisional application No. 61/538,529, filed on Sep. 23, 2011.

(51) Int. Cl. A61K 8/67; A61K 8/64; A61Q 19/06; A61K 8/58

(52) U.S. Cl. CPC A61K 8/676; A61K 8/58; A61K 8/64; A61Q 19/06
USPC 514/18.8

(57) ABSTRACT

A skin cream and method for making and applying the skin cream having, e.g., a copper component, an ascorbic acid component, a lipid component and a peptide component. The peptide component can include palmitoyl pentapeptide (KTTLS) or hydrolized serecin. The copper component can be a copper solubilizing formulation. The copper solubilizing formulation can include one of (I) copper and a copper-complexing peptide and/or (ii) a copper-complexing protein. The combination of copper and the ascorbic acid component can prevent or reduce a destabilization of the ascorbic acid component.

### FIELD OF THE DISCLOSURE

The present disclosure relates generally to a skin cream and a method for making thereof, and more specifically, relates to a skin cream that is used to prevent and/or reduce stretch marks.

### BACKGROUND INFORMATION

Stretch marks, or striae as they are called in dermatology, are a form of scarring on the skin with an off-color hue, and can be caused by tearing of the dermis and a break down of the collagen bed in the skin. Stretch marks are often the result of the rapid stretching of the skin associated with rapid growth (e.g., common in puberty) or weight gain such as muscle building, or rapid increase in fat or, in some cases, severe pulling force on skin that overcomes the dermis's elasticity. Stretch marks may also be influenced by hormonal changes.

Stretch marks are most commonly associated with pregnancy. It is estimated that between 75% and 90% of women will develop some degree of stretch marks during their pregnancy. Pregnancy stretch marks usually appear during the sixth or seventh month, primarily during the 3^{rd} trimester as the skin tends to be subjected to higher levels of stretching forces.

Various treatments have been proposed to remove stretch marks, or at minimum, improve their appearance. Current treatments include laser removal, surgery (e.g., tummy tuck), and skin cream. The problem with prior treatments is that they are only used to remove or diminish existing stretch marks, and not to prevent the formation of stretch marks. Their success is limited as the removal of an existing stretch mark can often be ineffective.

Copper is a trace element that has been determined to be implicated in various biological functions of the body. Copper has been shown to have healing, anti-inflammatory, collagen building as well as antimicrobial effects. A significant problem with Copper, however, is that is not easily absorbed into the skin, especially when applied topically. Because of its difficulty in being absorbed into the skin, copper has been infrequently used in the industry with limited success. For example, copper EDTA and Copper sulfate have been used as chelating and astringent agents, as well as colorants.

A further problem with copper is that it can cause stability problems in cosmetic formulations. Copper can cause crystallization, color fade, decomposition, product separation, as well as various other problems when combined into a cosmetic formula. Therefore, copper is known not to be used with other healing agents as a combination that includes copper can render each component useless for it intended function.

Ascorbic Acid (e.g., Vitamin C) is known for promoting the proliferation of collagen, and has been demonstrated to convert prolein residues into hydroxyproline during collagen biosynthesis. Additionally, as a vitamin, it is a required trace nutrient and antioxidant. In general, vitamin C can be very difficult to stabilize. To help circumvent the stability problems with vitamin C, derivatives of vitamin C such as disodium ascorbyl sulfate, ascorbyl palmitate, ascorbyl glucoside, magnesium ascorbyl palmitate, tetrahexyldecyl ascobate, ethyl ascorbate, can be used, as these derivatives can improve on the stability of vitamin C to varying degrees. However, when combined with a redox metal, such as copper, the enhanced stability of these derivatives can be greatly reduced or even lost.

Peptides can be utilized to induce the body to perform certain functions. For example, trace fragments of certain proteins (e.g., peptides) can be recognized as signals to perform biological functions. Matrixyl™ (e.g., Palmitoyl Pentapeptide-4), can produce a signal to the body that there has been collagen decomposition and that the biosynthesis of new collagen is required. The Palmitoyl group of this compound can be synthetically added to enhance penetration of the peptide. Upregulex™ (e.g., Serecin Peptides) can also be used to boost collagen. Peptides can be delivered in a liposome (e.g., more phospholipids) which can be used to complex Copper.

Lipids are the largest non-aqueous components of cell membranes. They can control the integrity and fluidity of the membrane. Lipids, especially the class of lipids called ceramides, can consitite the intercellular matrix, the “mortar”, which can hold skin cells together.

Copper is a metal that is generally easily oxidized or reduced. Its common oxidation states can include the cuprous (I), cupric (II), and metallic (0) states. Ascorbic acid is also extremely easily oxidized, reduced, or thermally decomposed. As such, copper and vitamin C are generally known in the art to be incompatible with each other and are generally not formulated together.

Various skin creams have been proposed for use with stretch marks; however, these skin creams are not formulated to facilitate the easy or effective absorption into the skin. Many of the current skin creams are not able to penetrate the layer of dead skin, the outermost layer of skin. Additionally, many of the skin creams use ingredients that while themselves may be helpful in the reduction of stretch marks, the combination can interact to render the individual components ineffective. Therefore, for the reduction of stretch marks, many people use multiple skin creams at a time, having to wait for the previous skin cream to be absorbed before applying the next cream. This can be ineffective as the person needs to keep close track of the individual skin creams applied and when they were applied.

Thus, a single skin cream can be desirable that facilitates the easy absorption of the individual ingredients, including copper and vitamin C, while keeping the ingredients separate to maintain their stability and prevent their interaction. It can also be desired that the skin cream be applied prior to the formation of the stretch mark in order to limit the damage done by the stretch mark or prevent the stretch mark entirely.

### SUMMARY OF EXEMPLARY EMBODIMENTS

Exemplary embodiments of the present disclosure relate to skin cream, method for preparing a skin cream, and method for applying a skin cream which can include a copper component, a vitamin C component, a lipid component and a peptide component. The peptide component can include palmitoyl pentapeptide (KTTLS) or hydrolized serecin. The copper component can be a copper solubilizing formulation. The copper solubilizing formulation can include one of at least copper and copper-complexing peptide or a Copper-complexing protein. The combination of copper and vitamin C does not destabilize the vitamin C.

In some exemplary embodiments, copper solubilizing formation can comprise copper gluconate. In certain exemplary embodiments, the skin cream can further include one of at least water, Tetrasodium EDTA, Butylene Glycol, Glycerin, Ketrol CG-T, Pationic ISL, Biophilic H, Arlacel 165, Softisan 649, Shea Butter, Dow Corning 200 Fluid, 100 cst, Sonnenatural, Myritol 318, Macadamia Nut Oil, Cetyl Alcohol, Wickenol 171, Dow Corning 1403 Fluid, Evening Primrose Oil, DC HydroGlide, Matrixyl 3000, D-Panthenol, BVOSC, Vitamin E Acetate, Diocide, Deionized Water, Potassium Sorbate, Sodium Benzoate. In some exemplary embodiments, the skin cream can further include one of at least dimethicone and dimethiconol, caprylic/capric triglyceride or Olus.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a table providing an exemplary list of ingredients that can be used in the exemplary skin cream;

FIG. 2 is an exemplary method for creating the exemplary skin cream according to an exemplary embodiment of the present disclosure;

FIG. 3 is a table providing an exemplary ingredient list sorted by phase;

FIG. 4 is an exemplary method for combining the exemplary ingredient list from FIG. 3 according to an exemplary embodiment of the present disclosure; and

FIG. 4 is an exemplary method for applying the exemplary skin cream according to an exemplary embodiment of the present disclosure.

Throughout the drawings, the same reference numerals and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the particular embodiments illustrated in the figures.

### DETAILED DESCRIPTION

Exemplary embodiments of the present disclosure may be further understood with reference to the following description and the related appended drawings. The exemplary embodiments of the present disclosure relate to an exemplary skin cream, an exemplary method for making the exemplary skin cream, and an exemplary method for applying the exemplary skin cream, that can prevent or reduce stretch marks of the skin.

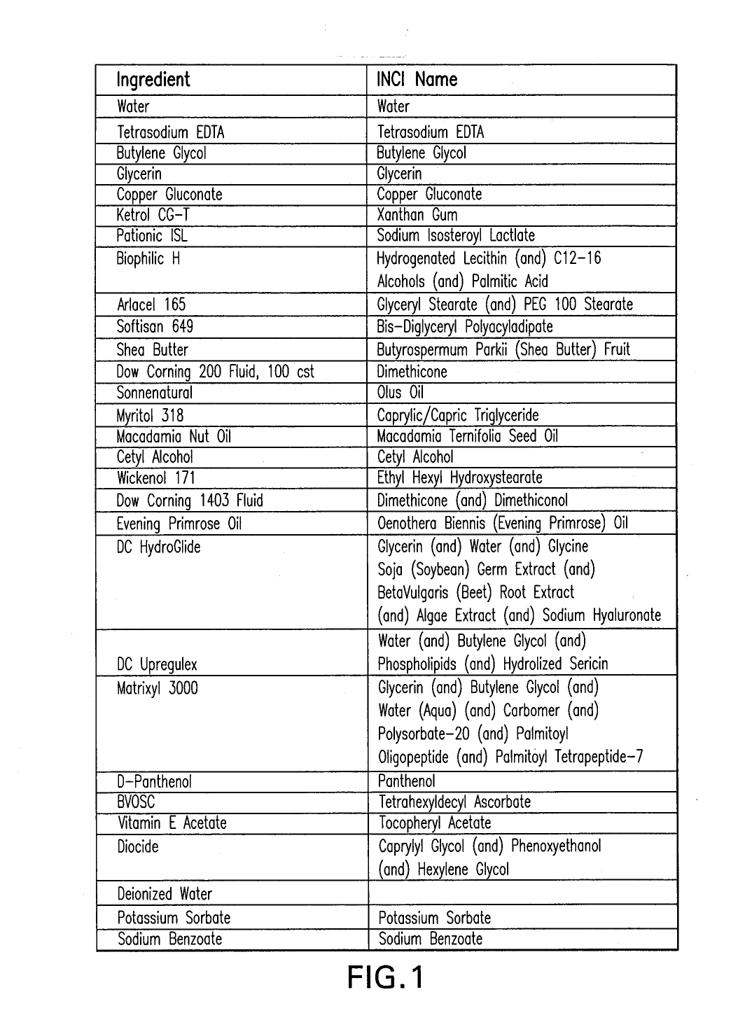

FIG. 1 shows a table providing an exemplary ingredient list according to an exemplary embodiment of the present disclosure. The combination of ingredients from this exemplary ingredient list can be used to prevent the formation of stretch marks, or the remove stretch marks once they have been formed. While all of the exemplary ingredients can be used to reduce or prevent stretch marks, the combination of a) copper, b) ascorbic acid, c) a lipid and d) a pentapeptide such as palmitoyl pentapeptide (KTTLS) and/or hydrolized serecin can provide the greatest effect on the reduction of the stretch marks. A combination of these four ingredients can have a synergistic effect to enhance the absorption of all of the ingredients into the skin where previously the separate components (e.g., copper and vitamin C) were not easily absorbed into the skin, or were destabilized by a combination of these components.

Copper can be an essential trace metal, which can increase dermal granulation tissue formation in wounds, hastening skin repair. Copper dependent enzymes (e.g., amine oxidases) are increased during wound healing. Lysyl oxidase catalyses the formation of aldehyde crosslinks and acts on collagen and elastin formation during wound healing. Copper is implicated in the increase of cell proliferation through the mechanism of metallothioneins. Copper also has an anti-inflammatory action having antioxidant properties. It can inhibit the action of tyrosinase, thus decreasing the formation of melanin. Copper salt derivatives can be used to thicken skin, and improve keratinocyte and dermal fibroblast function. It also has anti-infective activity. It should be noted that can be used in any suitable form including copper salt, copper gluconate, elemental copper, and any other complexed copper. Copper levels can be between 0.0001% to 2%, although not limited thereto.

In various exemplary embodiments, a copper solubilizing formulation can be provided to increase the absorption of copper. For example, a skin-absorbable copper-complexing peptide or protein can be formed. In order to enhance the absorption of copper, copper can be combined with a peptide in order to form a stable bond that can more easily be absorbed into the skin than copper by itself. This exemplary combination of active peptides to complex copper can facilitate the coexistence with ascorbic acid and ascorbic acid derivatives (e.g., vitamin C) where previously these components could not coexist. Additionally, copper and peptide moieties, which can be delivered together, e.g., one carrying the other, can bring both active ingredients, where the stretch marks are to be prevented, and where they can more effectively perform their function.

In an exemplary an embodiment of the exemplary skin cream, copper Gluconate can be combined with the DC Upregulex, which can complex Copper to form Copper-complexing peptides, which can enhance the absorption of copper into the skin.

Lipids can be used to increase the softness, fluidity, and hydration of the skin. This can facilitate an enhanced absorption of copper/peptide bond into the skin in order to prevent the formation of stretch marks, or to reduce previously formed stretch marks. The use of lipids and liposomes can enhance the skin's ability to assimilate vitamin C, copper, and peptides by directly acting as a protective reservoir on the skin's surface, by forming a “second skin”. The lipids can include any suitable class of cosmetic emulsifiers including ethoxylated ethers and esters of fatty alcohols, phospholipids, fatty alcohols, fatty acids, or other derivatives of fatty alcohols, and preferably have levels between about 0.1 to 50%, although not limited thereto.

Vitamin C can be used to stimulate collagen formation (e.g., Type 1 and Type 3 collagen 3-4-fold over control levels). Vitamin C is the rate limiting co-factor for this process. It can also lighten hyperpigmentation. The above exemplary combination of Copper and a peptide, can facilitate vitamin C to be intact (e.g., not destabilized) and participate in the collagen boosting process together with copper and the peptide. Vitamin C level can be between about 0.001% to 50%, although not limited thereto.

Palmitoyl pentapeptide, hydrolized serecin, and/or other peptides can be used to increase collagen formation. The increased dermal strength from increased collagen production, and ‘stretchability’ or elasticity from increased elastin production, leads to the prevention of stretch marks when the skin is subjected to increased tension and/or is under hormonal influences. Peptide levels can be between about 0.01% to 5%, although not limited thereto.

Emolliation can also be enhanced by various components including glycerin, Shea butter, dimethicone and dimethiconol, caprylic/capric triglyceride, Olus and Macadamia Oil, although not limited thereto. Vegetable oils contain essential fatty acids and can be unsaturated antioxidant lipids. Phospholipids, which are “skin type” lipids, can emolliate and replenish the lipids in the skin. Collectively such emolliation can serve to minimize the physical stress forces imposed upon the skin.

Panthenol can emolliate the skin and can also be a humectant, which can hold water by electrostatic forces. Panthenol can also increase cell proliferation.

The stability of this formulation, by combining copper with a pentapeptide, protects the active ingredients, such as copper and vitamin C, from inhibiting each other, as well as reducing the risk of oxidation, which would reduce the benefit of the cream.

Collectively, the ingredients in the exemplary skin cream, due to the nature of their chemical properties, can minimize damage to the skin, especially “stressed” skin. By providing a combined antioxidant protection, which can help minimize the effects of matrix metalloproteinase (MMP) overexpression, and overactivity, this can help mitigate the breakdown of the very structural proteins the exemplary skin cream enhances through various other exemplary ingredients.

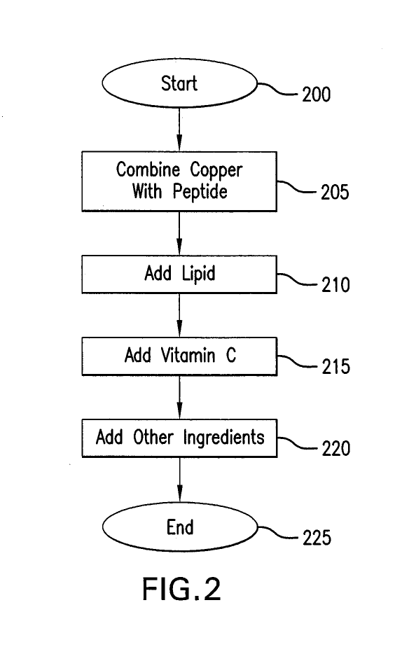

FIG. 2 illustrates an exemplary method to form the exemplary skin cream according to an exemplary embodiment of the present disclosure. The exemplary method begins at procedure 200. At procedure 205, copper, in any suitable form, can be combined with a peptide to complex copper. At procedure 210, a lipid can be added to the combination of copper and the peptide. At procedure 215, vitamin C can be added to the exemplary compound. At procedure 220, additional ingredients that can aid in the prevention of stretch marks and/or the reduction of existing stretch marks, can be added. The exemplary method can end at procedure 225.

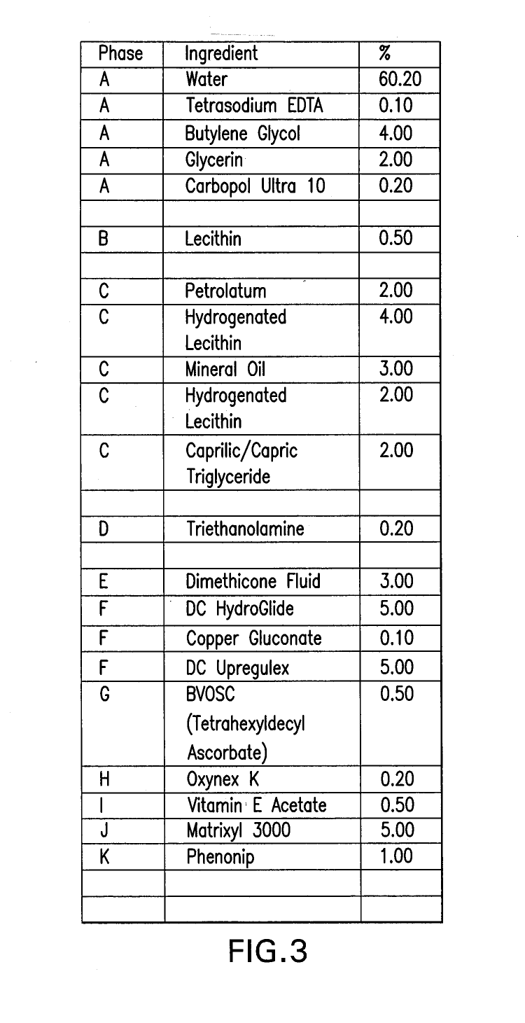

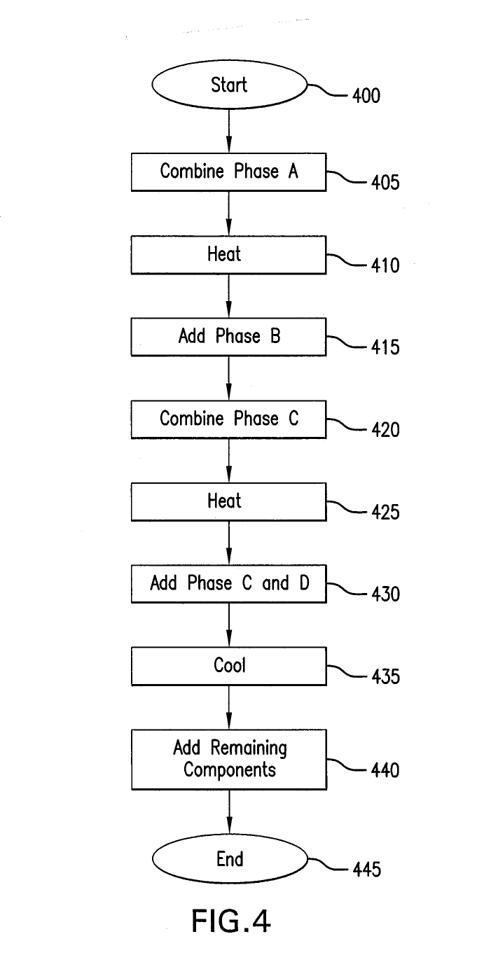

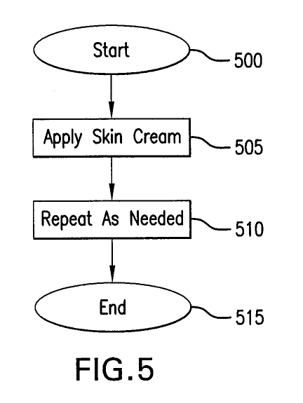

FIG. 4 illustrates a further exemplary method for creating the exemplary skin cream using the exemplary phase ingredient list from FIG. 3 according to an exemplary embodiment of the present disclosure. The exemplary method begins at procedure 400. At procedure 405, the phase A ingredients can be prepared in a tarred main vessel prepare by adding water, dissolving EDTA, 1,3 Butylene Glycol and Glycerin. With rapid mixing Carbopol Ultra can be dispersed and the entire mixture can be mixed until it is uniform. At procedure 410, the mixture can be heated to approximately 80 degrees Celsius. At procedure 415, the components from phase B can be added to the mixture, maintaining the temperature while mixing. At procedure 420, the components from phase C can be separately combined in a secondary vessel. At procedure 425, the phase C mixture can be heated to approximately 80 degrees Celsius, and the mixture can be mixed until it is uniform. At procedure 430, the phase C mixture can be added to the phase A and phase B mixture to form an emulsion, and once the mixture is uniform, the phase D components can be added. At procedure 435, the entire mixture can be cooled to approximately 35-40 degrees Celsius. At procedure 440, any remaining components can be added and mixed until the mixture is uniform. Water can be added to the mixture to replace any water that evaporated during the mixing process. At procedure 445, the method for creating the exemplary skin cream ends and a stable formulation can be achieved.

FIG. 5 illustrates an exemplary method for applying the exemplary skin creatin according to an exemplary embodiment of the present disclosure. At procedure 500, a user of the exemplary skin cream desires to prevent the formation of the stretch marks, and/or reduce the existences of previously formed stretch marks. At procedure 505, the user applies the exemplary skin cream to the areas that the user wishes to prevent the formation of stretch marks, and/or areas in which stretch marks have already formed. At procedure 510, the application of the exemplary skin cream is repeated as desired by the user. At procedure 515, the exemplary method of the exemplary skin cream ends.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. Further, to the extent that the prior art knowledge has not been explicitly incorporated by reference herein above, it is explicitly incorporated herein in its entirety.

What is claimed is:

1. A skin cream formulation comprising:
a mixture of a copper component, an ascorbic acid component, a lipid component, and a peptide component.

2. The skin cream of claim 1, wherein the peptide component comprises one of at least palmitoyl pentapeptide (KTTLS) or hydrolized serecin.

3. The skin cream formulation of claim 1, wherein the copper component is in a copper solubilizing formulation.

4. The skin cream of claim 3, wherein the copper solubilizing formulation comprises one of (i) copper and a copper-complexing peptide, or (ii) a copper-complexing protein.

5. The skin cream of claim 4, wherein the copper solubilizing formation comprises Copper gluconate.

6. The skin cream of claim 1, further comprising at least one of water, Tetrasodium EDTA, Butylene Glycol, Glycerin, Ketrol CG-T, Pationic ISL, Biophilic H, Arlacel 165, Softisan 649, Shea Butter, Dow Corning 200 Fluid, 100 cst, Sonnenatural, Myritol 318, Macadamia Nut Oil, Cetyl Alcohol, Wickenol 171, Dow Corning 1403 Fluid, Evening Primrose Oil, DC HydroGlide, Matrixyl 3000, D-Panthenol, BVOSC, Vitamin E Acetate, Diocide, Deionized Water, Potassium Sorbate, or Sodium Benzoate.

7. The skin cream of claim 1, further comprising one of (i) dimethicone and dimethiconol, (ii) caprylic/capric triglyceride or (iii) Olus.

8. The skin cream of claim 1, wherein the combination of the copper component and the ascorbic acid component avoids a destabilization of the ascorbic acid component.

9. The skin cream of claim 1, wherein the ascorbic acid component is vitamin C.

10. The skin cream of claim 1, wherein the skin cream prevents a formation of at least one stretch mark.

11. The skin cream of claim 1, wherein the skin cream substantially reduces a stretch mark.

12. A method of preparing a skin cream to prevent or reduce a formation of at least one stretch mark, comprising:
combining a Copper component, an ascorbic acid component, a lipid component and a peptide component.

13. The method of claim 12, wherein the peptide component comprises one of palmitoyl pentapeptide (KTTLS) or hydrolized serecin.

14. The method of claim 12, wherein the copper component is in a Copper solubilizing formulation.

15. The method of claim 14, wherein the copper solubilizing formulation comprises one of (i) copper and a copper-complexing peptide, or (ii) a copper-complexing protein.

16. The method of claim 14, wherein the copper solubilizing formation comprises Copper gluconate.

17. The method of claim 12, further comprising adding at least one of water, Tetrasodium EDTA, Butylene Glycol, Glycerin, Ketrol CG-T, Pationic ISL, Biophilic H, Arlacel 165, Softisan 649, Shea Butter, Dow Corning 200 Fluid, 100 cst, Sonnenatural, Myritol 318, Macadamia Nut Oil, Cetyl Alcohol, Wickenol 171, Dow Corning 1403 Fluid, Evening Primrose Oil, DC HydroGlide, Matrixyl 3000, D-Panthenol, BVOSC, Vitamin E Acetate, Diocide, Deionized Water, Potassium Sorbate, or Sodium Benzoate.

18. The method of claim 12 further comprising adding one of at least dimethicone and dimethiconol, caprylic/capric triglyceride or Olus.

19. The method of claim 12, wherein the combination of copper component and the ascorbic acid component does not destabilize the ascorbic acid component.

20. The method of claim 12, wherein the ascorbic acid component is vitamin C.

21. The method of claim 12, wherein the skin cream further substantially reduces at least one stretch mark.

22. A method for reducing a formation of at least one stretch mark comprising:
applying a skin cream containing a copper component, an ascorbic acid component, a lipid component and a peptide component.

23. The method of claim 22, wherein the peptide component comprises one of palmitoyl pentapeptide (KTTLS) or hydrolized serecin.

24. The method of claim 22, wherein the copper component is provided in a copper solubilizing formulation.

25. The method of claim 24, wherein the copper solubilizing formulation comprises one of (i) copper and a copper-complexing peptide, or (ii) a copper-complexing protein.

26. The method of claim 25, wherein the copper solubilizing formation comprises copper gluconate.

27. The method of claim 22 further comprising applying the skin cream after at least one stretch mark has formed.

28. The method of claim 22, wherein the ascorbic acid component is vitamin C
